(19) United States
(12) Patent Application Publication
(10) Pub. No.: US 2009/0214498 A1
(43) Pub. Date: Aug. 27, 2009

(54) ANTIMICROBIAL PEPTIDES AND BACTERIAL STRAINS THAT PRODUCE THEM

(75) Inventors: Paul Ross, Co. Cork (IE); Catherine Stanton, Co. Cork (IE); Colin Hill, Cork (IE); Ger Fitzgerald, Co. Cork (IE)

Correspondence Address: CHALKER FLORES, LLP, 2711 LBJ FRWY, Suite 1036, DALLAS, TX, 75234, US

(73) Assignee: TEAGASC- NATIONAL DIARY PRODUCTS RESEARCH CENTRE, Fermoy (IE)

(21) Appl. No.: 12/094,445

(22) PCT Filed: Nov. 20, 2006
(86) PCT No.: PCT/IE06/00130

§ 371 Date: Oct. 8, 2008

(30) Foreign Application Priority Data
Nov. 21, 2005 (IE) S20050773

(51) Int. Cl. A61K 35/74; C07K 7/00; C12N 1/21; C12N 1/20; A61K 38/10; A61K 38/08; A23C 9/12; A23L 1/28

(52) U.S. Cl. 424/93.45; 530/328; 530/327; 435/252.3; 435/252.9; 514/16; 514/14; 426/61; 426/580

(57) ABSTRACT

The present invention relates generally to the field of health promoting agents, in particular antimicrobial agents and provides antimicrobial peptides and bacterial strains that provide the antimicrobial peptides. In one aspect, the invention provides a biologically pure culture of Lactobacillus acidophilus, strain DPC6026, a sample of which has been deposited at the National Collection of Industrial and Marine Bacteria, Aberdeen, Scotland on 18th Nov. 2005 under the accession number NCIMB 41354, or a derivative or mutant thereof capable of producing from milk or a milk product, peptides having antimicrobial activity.

### FIELD OF THE INVENTION

The present invention relates generally to the field of health promoting agents, in particular antimicrobial agents and provides antimicrobial peptides and bacterial strains that provide the antimicrobial peptides.

### BACKGROUND TO THE INVENTION

Significant health problems can occur in both humans and animals with infections by pathogenic bacteria. In particular, infections by pathogenic strains such as E. coli, E. sakazakii, Streptococcus mutans and Listeria innocua can be extremely debilitating. It has been recognized that there exists a need to provide antimicrobial agents to pathogenic bacteria.

Increasing resistance of pathogenic bacteria to antibiotics is a major problem and the search for novel drugs continues (35). Multi-drug resistant E. coli, Klebsiella spp., as well as strains of E. sakazakii have increased at an alarming rate and are frequently associated with immunocompromised hosts for whom they may have devastating effects (6, 14). Enterobacter species are recognised as increasingly important pathogens in recent years due to their innate resistance to older antimicrobial agents and their increased association with nosocomial infections (41). For over three decades, E. sakazakii has been recognised as the cause of a distinctive syndrome of meningitis in neonates (20). A mortality rate of 40-80% has been reported and there is increased compelling evidence ((20), (33), (34)) that milk based infant formulas serve as a reservoir of infection. Current industry efforts to control E. sakazakii infection have focused on improving manufacturing hygiene practices and end product testing for the organism (34). One possible route to identifying candidate novel antimicrobial peptides is in the investigation of milk proteins.

Milk proteins are a rich source of bioactive peptides, reported to have a range of health-promoting properties including angiotensin-I converting enzyme (ACE) inhibition, opioid, immunomodulation, antithrombosis, and antimicrobial activity (9). Bioactive peptides are present in milk proteins such as casein and whey in an encrypted form, stored as propeptides or mature C-terminal peptides (13, 19) with proteolysis required for their release (11). The best characterised of these functional peptides are derived from casein and have been shown to have effects in the cardiovascular system, mainly via ACE inhibition and consequently have antihypertensive features (43). Maruyama and Suzuki (28) reported that tryptic hydrolysates of casein inhibited the in vitro activity of ACE. Pihlanto-Leppala et al. (37) studied the ACE-inhibitory activity of peptides from whey and casein proteins fermented with lactic acid bacteria (LAB) and hydrolysed afterwards with digestive enzymes. Peptides identified were αₛ₁-casein f(142-147), f(194-199) and β-casein f(108-113) as well as two ACE-inhibitory peptides from whey (37). In addition, a range of antibacterial peptides have been isolated from bovine αₛ₂-casein (23). These include: Casocidin-1, f(164-179): KTKLTEEEKN RLNFLKKISQ RYQKFALPQY LKTVYQHQK, f(164-179): KKTKLTEEEK NRLNFL, and f(187-207): QKFALPQYLK TVYQHQKAMK Q.

These three peptides are derived from treating bovine milk with acetic acid and calcium sulphate to form a mixture which is then heated to form a precipitate and a supernatant. The supernatant is removed and treated with a cation-exchange resin. The peptides are eluted using chromatographic procedures and the active peptide casocidin-1 is released using digestive proteases. The cost of production is one possible limitation to the widespread application of these three peptides. In particular, Casocidin-1 has been shown to be bactericidal against Escherichia coli and Staphlylococcus carnosus in vitro (51).

However, no known bacterial strain produces casocidin-I directly, meaning that production of casocidin-I requires the use of enzymes and chemicals, which add to the cost of production. Further information can be found in U.S. Pat. No. 6,579,849 entitled “Antibiotic peptides from bovine milk”. No limitations on the activity of these peptides are mentioned in the above patent but Zucht et al., (FEBS Letters 372 (1995) 185-188), describes the spectrum of inhibition of these peptides and it appears that only Gram-negative bacteria are affected by these peptides, meaning that Gram-positive infections and contamination could not be treated with the peptides of U.S. Pat. No. 6,579,849.

Proteinases of microbial origin, including LAB, by their specificity and activities play a primary role in the generation of peptides from casein and potentially the release of antimicrobial peptides (29). Consequently, during fermentation the microorganisms producing these proteinases must be able to degrade milk proteins such as casein and whey in order to grow in milk. Casein degradation and subsequent utilization of the degradation products by LAB requires a complex proteolytic system (9). Due to the highly proteolytic nature of LAB such as Lactococcus lactis (22, 38) and Lactobacillus helveticus (27, 50), and their need to degrade milk proteins for growth in milk, their use as starter cultures for the generation of bioactive peptides has been reported (12, 25, 32, 45). Miniervini et al. (30) used a proteinase from Lactobacillus helveticus PR4 to produce bioactive peptides exhibiting antimicrobial activity from sodium caseinates of the milk from six species of mammals. An antimicrobial peptide corresponding to human β-casein f(184-210) was produced by hydrolysis of human sodium caseinate with a partially purified proteinase of L. helveticus PR4 and showed antimicrobial activity against several Gram-positive bacteria and also against E. coli (30). Characterization of the peptides produced during casein degradation has been described for L. helveticus (11, 49) and to a lesser extent for L. casei (7). Also, the cell wall bound proteinase of L. delbrueckii subsp. lactis ACA-DC 178 has been characterised and its specificity for β-casein has been documented (45).

Few casein derived antibacterial peptides have been reported and those that have been described result from the action of enzymes on the mature C-terminal peptide, leading to release of the active peptide sequence (43). The first antimicrobial peptides of casein origin were identified by Hill et al. (17) who isolated antibacterial glycopeptides, known as casecidins, following proteolysis of casein with chymosin at pH 6.4. Isracidin, a positively charged antimicrobial peptide, αₛ₁-casein peptide residue 1-23 with the primary amino acid structure R₁PKHPIKHQGLPQEVLNENLLRF₂₃ (17), was shown to have a broad spectrum of activity against both Gram-positive and Gram-negative bacteria (23) and to prevent mastitic infections in sheep and cows (12).

There is also a persistent problem in that pathogenic bacteria over time develop resistance to existing antimicrobial compounds. There is therefore an ongoing need for the development of new antimicrobial peptides in order to continually meet the challenge presented by resistant strains. In some situations, novel antimicrobial compounds may require more onerous application methodologies, such as increased concentration, increased number of applications, or may only be efficacious against a narrower range of target species. Even in these circumstances, the novel antimicrobial is still advantageous over the existing compounds, due to the problem of increased resistance. Furthermore, it would be of great benefit to be able to provide a stain of bacteria to produce such antimicrobial peptides, as this can firstly offer an economical method of producing the peptide and secondly that can provide a means to produce the peptides in situ situations.

### OBJECT OF THE INVENTION

One of the objects of the present invention is to provide a novel stain of bacteria that produces at least one antimicrobial peptide. It is a further object of the invention to provide at least one antimicrobial peptide. A further object is to provide an antimicrobial peptide for use against multiple drug resistant organisms. It is also an object of the invention to provide a foodstuff with improved safety containing antimicrobial agents. It is a further object of the invention to provide a method of ACE inhibition. It is also an object of the invention to provide a milk formula with improved antimicrobial properties.

### SUMMARY OF THE INVENTION

In one embodiment, the invention provides an antimicrobial compound comprising one or more peptides selected from the group consisting of SEQ ID NO: 1 (IKHQHPQE), SEQ ID NO: 2 (SDIPNPIGSENSEK), and SEQ ID NO: 3 (VLNENLLR). In an alternative embodiment, the invention provides a biologically pure culture of Lactobacillus acidophilus, strain DPC6026, a sample of which has been deposited at the National Collection of Industrial and Marine Bacteria, Aberdeen, Scotland on 18^{th} Nov. 2005 under the accession number NCIMB 41354, or a derivative or mutant thereof capable of producing from milk or a milk product, peptides having antimicrobial activity.

The invention also provides for proteins produced by DPC 6026. In particular, the invention provides for novel protyolitic enzymatic proteins produced by DPC6026 that are capable of producing one or more of peptides capable of producing anti-microbial peptides from milk. These peptides may be those with sequences substantially equal to one or more of the group consisting of SEQ ID NO: 1, SEQ ID NO: 2 and SEQ ID NO: 3 from milk or milk products.

The invention also provides for a method of producing an anti-microbial peptide from milk or milk products comprising adding to milk or a milk product, one or more of the group consisting of a biologically pure culture of Lactobacillus acidophilus strain DPC6026, or a derivative or mutant thereof capable of producing a from milk or a milk product, peptides having antimicrobial activity, a cell-free culture supernatant which is obtained from the DPC6026 culture, or a fraction thereof, and a protein produced by DPC6026, wherein the protein is capable of producing one or antimicrobial peptides from milk or milk products.

The invention also provides for a method of producing one or more proteins from DCP6026 that have proteolytic, catalytic and/or enzymatic properties that are capable of producing at least one of SEQ ID NO: 1, SEQ, ID NO: 2 or SEQ ID NO: 3 from milk or milk products.

An antimicrobial composition comprising one or more selected from the group consisting of an antimicrobial compound as claimed in claim 1, a culture as claimed in claim 2, a supernatant as claimed in claim 3 and a protein as claimed in claim 4.

The invention also provides a cell-free culture supernatant, or a fraction thereof, which is obtained from strain DPC6026, or an antimicrobial composition comprising one or more of the peptides, compounds, supernatant or strain as described above, together with a suitable carrier or diluent.

The invention also provides for the use of the compounds, peptides, strain, proteins, supernatant or composition as described above in the control of microbial infections or microbial contamination. Aspects of the invention provide methods of preventing, or treating or limiting microbial infection or microbial contamination comprising administering one or more of the compounds strain, supernatant or composition of the invention to an animal or human. The microbial infection or microbial contamination may be a contamination or infection caused by one or more of the group consisting of Escherichia coli, E. sakazakii, Streptococcus mutans, Listeria innocua, Klebsiella spp and Staphylococcus carnosus. In particular, the infection or contamination may be mastitis.

In alternative embodiments, the infection or contamination maybe meningitis, and may be meningitis in neonates. In some embodiments the compound, peptides, strain, supernatant or composition may be provided in a milk-based formula. Thus, the peptides of the invention can be used as an in built protection system in the manufacture of infant formula products. One or more of the peptides may be particularly efficacious against Enterobacter Sakazakii Contaiminant.

Due to the diverse nature of bioactive peptides in terms of structure, spectrum of activity and potency (39) these peptides are prime targets for potential new drug design. The possibility of using antimicrobial peptides such as SEQ ID NO: 1 (IKHQGLPQE) and SEQ ID NO: 3 (VLNENLLR) derived from milk proteins as an in-built mechanism of protection against pathogenic strains such as E. sakazakii 5920 (ATCC12868) may provide a useful approach for enhancing the safety of milk powders, such as those used in infant formula manufacture.

The invention also provides methods of inhibition of angiotensin-I converting enzyme (ACE), opioid modulation, immunomodulation, and antithrombosis, comprising treating the candidate animal or human with one or more of the compositions, compounds, supernatants, peptides, or strains of the invention.

The peptides, strains, compounds, compositions and supernatants of the invention can also be used in the treatment of bovine and ovine mastitis. SEQ ID NO: 1 and SEQ ID NO: 3 are similar to cleavage products of the antimicrobial peptide isracidin. SEQ ID NO: 2 may also be used. Isracidin has been documented as having a strong protective effect against S. aureus, Streptococcus pyogenes and L. monocytogenes when administered at doses as low as 10 μg per mouse and has also been used previously in the treatment of ovine and bovine mastitis.

The peptides, strains, compounds, compositions and supernatants of the invention can be used to contribute bitter flavours in the manufacture of bitter cheese flavours (Pederson et al., 1999, J. Bacteriology 181, 4592-4597) as the first 9 residues of αₛ₁-casein accumulate in cheese making and are responsible for the bitter flavours attributed to some cheeses (Fox et al., 1995, Chemistry of Structure-Function Relationships in Cheese (Malin, E. L., and Tunick, M. H., eds), Plenum Press, New York, pp 59-98).

The peptides, strains, compounds, compositions and supernatants of the invention can be used as preservatives of food or perishable goods against pathogenic Gram-negative and Gram-Positive bacteria due to the broad spectrum of activity of SEQ ID NO; 1, SEQ ID NO; 3 and SEQ ID NO: 2 as described.

L. acidophilus DPC6026 is a novel strain, isolated from the porcine small intestine and stocked in the Dairy Products Research Centre (DPRC), Teagasc Moorepark, Fermoy, Co. Cork. No bacterial strain has been documented showing the production of isracidin or its fragments previously. Isracidin was derived from αₛ₁-casein treated with chymosin (Hill et al. J of Dairy research 1974; 41: 147). This is advantageous economically as L. acidophilus DPC6026 in its natural state produces the three peptides IKHQGLPQE, VLNENLLR and SDIPIGSENSEK without the need for enzymes, avoiding additional expense to the fermentation process at an industrial level. The peptide sequence of Isracidin is more expensive than SEQ ID NO: 1, SEQ ID NO: 2 and SEQ ID NO: 3 to chemically synthesis due to its longer chain length SEQ ID NO: 1, SEQ ID NO: 2, SEQ ID NO: 3 each have shorter chain lengths and are therefore cheaper to chemically synthesis.

Isracidin αₛ₁-casein f(1-23) appears to maintain a relatively stable conformation in solution (Malin et al., Journal of Protein Chemistry, Vol. 20, 391-404). Conformationally stable peptides are known to be characteristic of allergens and this segment of αₛ₁-casein is known to be allergenic (Spuergin et al., 1996. Allergy 51, 306-312). The region of αₛ₁-casein surrounding residue 20 has been suggested to contain a significant allergenic epitope recognised by human IgE (Spuergin et al., 1996). In addition, SEQ ID NO: 3 may not present an allergenic epitope as it lacks residue 20 of αₛ₁-casein, which is thought to contain a significant allergenic epitope recognised by human IgE (Spuergin et al., 1996).

### Penetration In Vivo.

Isracidin has been shown to display a protective effect against Staphylococcus carnosus in vivo. SEQ ID NO: 1 and SEQ ID NO: 3 display a greater protective effect in vivo against pathogenic mastitis causing strains. It is hypothesised that the reason for this is that the shorter bioactive peptides display a better in vivo effect (Vermeirssen et al., British Journal of Nutrition (2004), 92, 357-366).

αₛ₁-casein f(1-9) (RPKHPIKHQ) has been shown to possess antihypertensive effect in the Spontaneously Hypertensive Rat (SHR) (Saito et al., 2000, J. Dairy Science, 83, 1434-1440). SEQ ID NO: 1 shares 100% homology with the last 4 amino acids (IKHQ). It also possesses proline, glutamine and glutamatic acid at the C-terminal end of the peptide. ACE activity is drastically slowed down by the presence of proline in the C-terminal tripeptides region (Vermeirssen et al., 2004, British Journal of Nutrition, 92, 357-366). This evidence would suggest that SEQ ID NO: 1 could possess ACE inhibitory activity and suggests a possible advantage of cleaving isracidin into two shorter peptides.

It is also hypothesised that under certain circumstances, the mechanism for Isracidin resistance mediated by an ‘antigenic’ region of the molecule not presented by either SEQ ID NO: 1 or SEQ ID NO: 3.

Other cationic antimicrobial peptides such as Lactoferricin B are known to have resistant strains such as Escherichia coli and Staphylococcus aureus due to protease production and this has been suggest as the main resistance mechanism against other cationic antimicrobial peptides such as magainins by E. coli and Staphylococcus aureus (Ulvatne et al., Journal of Antimicrobial Chemotherapy (2003) 50, 461-467). Cleaving isracidin into two of the peptides of the invention, SEQ ID NO: 1 and SEQ ID NO: 3, is likely to reduce the possibility of resistant strain development as fewer proteases can cleave these shorter sequences (as demonstrated using the computer program http://ca.expasy.org/cgi-bin/peptidecutter/peptidecutter.pl compared to isracidin.

### BRIEF DESCRIPTION OF THE DRAWINGS

FIG. 1: PFGE macrorestriction patterns for the restriction enzyme Apa I.

Pulse time was 1 to 15 s for the gel shown. Lane 1 contained low range PFG marker (A mixture of lambda DNA-Hind III fragments and lambda concatemers embedded in 1% LMP agarose), lanes 2 to 6, Lactobacillus acidophilus DPC6026, L. johnsonii DPC6092, L. salivarius DPC6027, L. animalis DPC6028 and L. delbrueckii sp. bulgaricus DPC6104.

FIG. 2: Proteolytic activity of GRAS strains.

High Performance Liquid Chromatography (HPLC) peptide profile of bovine sodium caseinate (A), and of its sodium caseinate hydrolysates after fermentation with Lactobacillus johnsonii DPC6092 (B), L. salivarius DPC6027 (C) and L. acidophilus DPC6026 (D). HPLC was monitored at 214 nm for 24 h. Sample 30 μl was loaded onto a Delta-Pak C-18 (600 mm×7.5 cm), flow rate of 1 ml/min. Proteolysis was carried out at room temperature. L. acidophilus DPC6026 showed almost complete breakdown of sodium caseinate (D).

FIG. 3

A. Reverse Phase-High Performance Liquid chromatography (RP-HPLC) chromatogram of sodium caseinate at pH 7 incubated with L. acidophilus DPC6026 for 24 h. Arrows indicate position of peptide fractions A1-45 and A1-54. RP-HPLC was carried out at room temperature and following the conditions shown in the materials and methods section.

B. Matrix Assisted Laser Desorption Ionisation-Time of Flight (MALDI-TOF) spectrum of fraction A1-45 (isolated using sodium caseinate as the substrate and the strain L. acidophilus DPC6026) recorded in the m/z region where peptides were detected. Peptide fraction A1-45 was found to contain a peptide of mass 1049.43, which was found after sequencing to be IKHQGLPQE, which corresponds to αₛ₁-CN f (21-29).

1. Inhibition of E. coli DPC6053 by the chemically synthesised peptide IKHQGLPQE identified in fraction A1-45 of the sodium caseinate hydrolysate with L. acidophilus DPC6026. A zone 2.5 cm in diameter was recorded using the agar well diffusion assay method.

2. Inhibition of Enterobacter sakazakii5920 (ATCC 128682) by the chemically synthesised peptide IKHQGLPQE identified in fraction A1-45 of the sodium caseinate hydrolysate with L. acidophilus DPC6026. A zone 2.0 cm in diameter was recorded using the agar well diffusion assay method.

C. Matrix Assisted Laser Desorption Ionisation-Time of Flight (MALDI-TOF) spectrum of fraction A1-54 (isolated using sodium caseinate as the substrate and the strain L. acidophilus DPC6026) recorded in the m/z region where peptides were detected. Peptide fraction A1-54 was found to contain a peptide of mass 970.119, which was found after sequencing to be VLNENLLR corresponding to αₛ₁-CN f (30-37).

1. Inhibition of E. coli DPC6053 by the chemically synthesised peptide VLNENLLR identified in fraction A1-54 of the sodium caseinate hydrolysate with L. acidophilus DPC6026. A zone 1.5 cm in diameter was recorded using the agar well diffusion assay method.

2. Inhibition of Enterobacter sakazakii8272 (NCTC8155) by the chemically synthesised peptide VLNENLLR identified in fraction A1-54 of the sodium caseinate hydrolysate with L. acidophilus DPC6026. A zone 2.2 cm in diameter was recorded using the agar well diffusion assay method.

FIG. 4:

A. Effect of isracidin at various concentrations on the OD₆₀₀ of E. coli DPC6053 (an overnight culture diluted 1:10 with LB broth) using the 96-well plate assay. Cecropin P1 is used as a control peptide.

Symbols: ▪, E. coli DPC6053 incubated with Cecropin P1 (0.52 mM).
-, E. coli DPC6053 control with no additions.
♦, E. coli DPC6053 incubated with isracidin (1.9 mM).
x, sterility control (LB broth with no additions).
, E. Coli DPC6053 incubated with isracidin at a concentration of 0.23 mM.
X, viability of E. coli DPC6053 incubated with isracidin (0.059 mM).

B. Effect of peptide IKHQGLPQE at various concentrations on the OD₆₀₀ of E. coli DPC6053 (an overnight culture diluted 1:10 with LB broth) using the 96-well plate assay.

Symbols: -, E. coli DPC6053 with no additions.
X, E. coli DPC6053 incubated with IKHQGLPQE (0.078 mM).
▪, E. coli DPC6053 incubated with Cecropin P1 (0.52 mM).
, E. coli DPC6053 incubated with IKHQGLPQE (0.15 mM).
•, E. coli DPC6053 incubated with IKHQGLPQE (0.31 mM).
♦, E. coli DPC6053 incubated with IKHQGLPQE (0.625 mM).
x, sterility control (LB broth with no additions).

C. Effect of peptide VLNENLLR at various concentrations on OD₆₀₀ of E. coli DPC6053 (an overnight culture diluted 1:10 with LB broth) using the 96-well plate assay.

Symbols: X, E. coli DPC6053 with no additions.
▪, E. coli DPC6053 incubated with VLNENLLR (0.22 mM).
-, E. coli DPC6053 incubated with VLNENLLR (0.45 mM).
♦, E. coli DPC6053 incubated with VLNENLLR (1.2 mM).
X, viability of E. coli DPC6053 incubated with Cecropin P1 (0.52 mM).
, E. coli DPC6053 incubated with VLNENLLR (2.5 mM).
▴, Sterility control—LB broth without additions.

### Materials and Methods

### Substrate and Chemicals

Peptides Indolicidin and Cecropin P1 and the enzyme Proteinase K were purchased from Sigma (Sigma Aldrich Chemie, Steinheim, Germany). Pimaricin was purchased from Merck (Merck, Darmstadt, Germany). The peptide Isracidin was synthesised by Peptide Protein Research Ltd., (Fareham, UK.). Maximum Recovery Diluent (MRD) was purchased from Oxoid (Oxoid Ltd., Basingstoke, England). Bovine sodium caseinate was from Dairygold (Mitchelstown, Cork, Ireland). Pulsed Field Certified agarose was from Bio-Rad Laboratories (Hercules, Calif. 94547, USA). Enzyme Apa I and the Low Range PFG Marker (NO350S) were purchased from New England Biolabs (Hertfordshire SG4 OTY, England). Low melting point agarose was purchased from Bio-Rad Laboratories (Richmond, Calif.).

### Microorganisms and Culture Conditions

L. acidophilus DPC6026, L. johnsonii DPC6092 and L. salivarius DPC6027 were isolated from the porcine small intestine (data unpublished) and stocked in the culture collection of Teagasc Dairy Products Research Centre, Cork, Ireland. These strains were propagated in MRS broth (Oxoid Ltd, Basingstoke, United Kingdom) anaerobically for 24 h at 37° C. Standard cultures were prepared by inoculation of 10 ml MRS broth with 10 μl of a frozen stock (−80° C.) and then incubated at 37° C. for 16-24 h. L. innocua DPC3306, E. sakazakii 5920 (ATCC12868), E. sakazakii 8272 (NCTC8155) purchased from the NCIMB (National Collection of Industrial and Marine Bacteria, Aberdeen AB24 3RY, Scotland), and E. coli DPC6053 (Teagasc Dairy Products Research Centre, Cork, Ireland) were employed as the test strains.

### Initial Screening for Proteolytic Activity on Skim Milk Agar (SMA) Plates

Two hundred and five bacterial isolates of porcine intestinal origin and 55 isolates of human adult and infant faecal origin (data unpublished) were screened for proteolytic activity using SMA plates as previously described (36). Clearing zones surrounding the spotted culture was indicative of protein degradation. Isolates producing zones ≧4.0 mm in diameter were selected for further study.

### High Performance Liquid Chromatography (HPLC)

Isolates showing proteolytic activity using SMA plates were inoculated (1% w/v) in a sodium caseinate solution (2.5% w/v) and incubated overnight anaerobically at 37° C. High Performance Liquid Chromatography using Delta-Pak C18 Column, (size; 600 mm×7.5 cm, Varian Chromatography Systems, Walnut creek, Calif., USA) was then performed on the resultant fermentates. The mobile phase was a binary mixture of acetonitrile and HPLC grade water (30% v/v) containing trifluoroacetic acid (0.1% v/v). The flow rate was 1 ml/min. Casein breakdown by both porcine intestinal isolates and infant faecal isolates was monitored by measurement of UV absorbance at 214 nm using a HP1100 diode array detector.

### Pulse-Field Gel Electrophoresis

Preparation of high-molecular weight DNA from each of the 15 isolates from MRS broth cultures was as previously described by Simpson et al. (42) except that 20 U of the restriction enzyme Apa I was used for the restriction digest of the plug slices.

16S rDNA Sequencing of Isolates to Speciate Strains

Fifteen isolates of pig intestinal and infant human and adult faeces, were shown to exhibit significant proteolytic activity and were subjected to 16s ribosomal DNA (rDNA) sequencing (Lark Technologies Inc., Essex, UK) to speciate the individual strains. Two 16S rDNA primers —CO1 for the 5′ end (5′-AGTTTGATCCTGGCTCAG-3′) and CO2 for the 3′ end (5′-TACCTTGTTACGACT-3′)—were used to generate an approximate 1.5 kb 16S rDNA product under Polymerase Chain Reaction (PCR) conditions described previously (42). This PCR product was partially sequenced using the CO1 primer (Lark). Comparison of the 16S rDNA sequences obtained by using the BLAST program allowed the assignment of a strain to a particular species. Generally, when 16S rDNA sequence similarity values exceed 97% using the BLAST program, the strains are considered to belong to the same species (44).

Large Scale Fermentation with GRAS Strains

Of the fifteen isolates exhibiting proteolytic activity, three genetically distinct strains, Lactobacillus acidophilus DPC6026, L. salivarius DPC6027 and L. johnsonii DPC6092 were used individually in further fermentations on the basis of their proteolytic capabilities and as they were all generally recognised as safe (GRAS). Three separate fermentations were performed in triplicate. The sodium caseinate substrate (2.5% w/v) was inoculated with each strain (1% w/v) and incubated at 37° C. for 24 h with mixing at 100 rpm at constant pH 7 maintained via addition of 0.1M NaOH. The fermentates were then heated to 80° C. to inactivate the cultures and subsequently filtered through a size-exclusion S1Y10 10 kDa spiral cartridge filter (Millipore Ltd., Hertfordshire, UK), to separate the peptides less than 10 kDa. Fractions containing these peptides were freeze-dried and stored at −20° C. until further use.

### Reverse-Phase HPLC Analysis of Fermentates

Peptides <10 kDa in size were separated from sodium caseinate hydrolysates using an RP-HPLC reverse-phase high performance liquid chromatography system containing a narrow-bore column (Nucleosil C18, 5 mm×250 mm: Varian Chromatography Systems, Walnut creek, Calif., USA) and an UV detector operating at 214 nm. Aliquots of the freeze-dried powders were diluted in distilled HPLC-grade water and filtered through a 0.45 μm filter (Millipore) and 30 mg/ml of the fermentate loaded onto the column. The mobile phase was a binary mixture of acetonitrile and HPLC grade water (100% v/v) containing trifluoroacetic acid (0.1% v/v). The content of acetonitrile in the mobile phase was increased linearly from 0 to 100% for 72 min at a flow rate of 1 ml/min. Peptides were detected using a detector operating at a wavelength of 214 nm. Solvents were removed from the collected fractions by evaporation using a centrivap console (Labconco Corporation, Kansas City, USA). The fractions were redissolved in 1 ml of distilled water prior to subsequent assays for antimicrobial activity.

### Determination of Protein Concentration

The protein concentration of the fractions was determined using the Biorad Protein Assay Method (24). Absorbance at 595 nm was determined and protein concentration reported as mg/ml.

### Antibacterial Activity

Preliminary characterisation of the antibacterial activity of peptides and fractions synthesised involved measurement of growth inhibition in a 96-well plate assay (1, 10) and utilisation of an agar diffusion method (16,39). The 96-well plate assay involved the use of controls of each peptide fraction alone (Cecropin P1 and indolicidin), as well as controls without peptide fractions (growth control). A control without bacteria (sterility control) was also included. The plates were incubated for 6 h at 37° C. and culture growth monitored hourly. Minimum inhibitory concentrations (MIC's) were taken as the lowest concentration without visible growth, measured by recording the OD₆₀₀, in a micro-titre plate reader. A well diffusion assay (16, 40) was used to detect antibacterial activity of purified and chemically synthesised peptides. These assays were performed in either Brain Heart Infusion (BHI), Luria Bertani (LB) or Nutrient agar (NA) seeded with 1 ml of an overnight culture of the indicator strains E. coli DPC6053, L. innocua DPC3306, E. sakazakii 5920 (ATCC12868) or E. sakazakii 8272 (NCTC8155) respectively. Wells, 4.6 mm in diameter, were cut into these agar plates, and 30 μl (0.162 mg/ml) of the peptide fraction placed in each well. Plates were stored at 4° C. for 4 h to permit radial diffusion of the peptide, incubated at 30° C. anaerobically or 37° C. aerobically for 24 h, and examined for zones of inhibition. The sensitivity of a strain to the peptides was scored according to the diameter of the zone of inhibition surrounding the well. The experiments were performed in triplicate and mean zone size calculated. Isracidin, Cecropin P1 for E. coli DPC6053, E. sakazakii 5920 (ATCC 12868) and E. sakazakii 8272 (NCTC8155) and Indolicidin against L. innocua DPC3306 were used as controls.

### Purification, Sequencing and Synthesis of Antimicrobial Peptides

Protein fractions exhibiting antibacterial activity were refractionated by RP-HPLC conditions as described above, and those fractions that inhibited growth of the indicator strain E. coli DPC6053 at a similar protein concentration to the commercial peptide Cecropin P1 were collected and the peptide composition analysed by Mass spectrometry (MS) with a matrix assisted laser desorption ionisation time of flight (MALDI-TOF) mass spectrophotometer (PE Biosystems Voyager-DE STR Biospectrometry Workstation, Aberdeen Proteome Facility) with a laser operating at 337 nm and an acceleration voltage of 20 kV. The amino acid sequences of the peptides in each fraction were determined after derivatization and Edman degradation. These steps were performed on a 494A protein sequencer (Applied Biosystems). All peptides identified (by MS analysis and amino acid sequencing) in each fraction exhibiting antimicrobial activity were subsequently chemically synthesised by Peptide Protein Research Ltd., (Fareham, UK). The purity of the synthesised peptides was greater than 95% as determined by HPLC analysis as certified by the manufacturer.

Treatment of Chemically Synthesised Peptides and Crude Fractions with Proteinase K

Chemically synthesised peptides and fractions exhibiting antimicrobial activity against E. coli DPC6053 were tested for susceptibility to proteinase K (Sigma) by incubation of proteinase K (2050 U/ml) with the peptide or fraction (0.554 mg/ml) in a 1:1 volume ratio at 4° C. for 6 h using the agar well diffusion assay (40). Plates were incubated at 37° C. for 24 h. The positive controls used were the peptides isracidin (Peptide Protein Research Ltd.) and Cecropin P1 (Sigma TM).

### EXAMPLES

One of the objectives of this study was to discover novel bioactive peptides from bovine casein released using the proteolytic capabilities of LAB of mammalian intestinal origin. 205 isolates obtained from the porcine small intestine and 55 isolates of human adults and infant faecal origin were used in this study.

Screening for Isolates with Proteolytic Activity

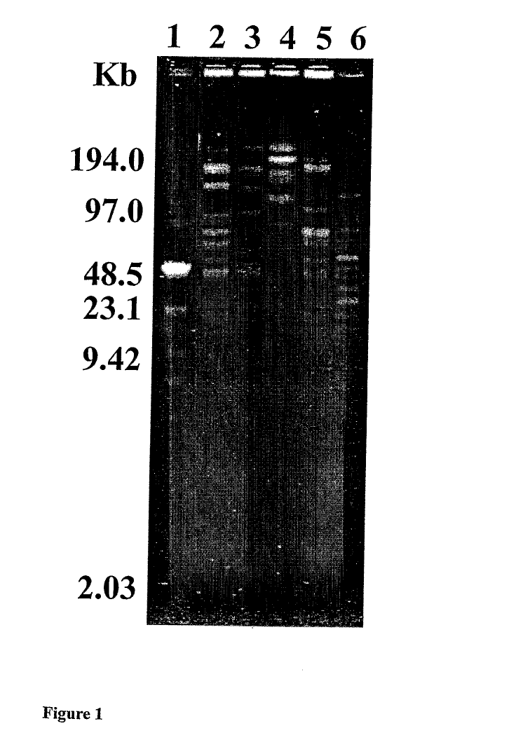

All 260 isolates on LAB agar plates were screened for proteolytic activity using SMA and sodium caseinate as substrates. 5 isolates of porcine intestinal origin and 10 isolates of human infant and adult faecal origin were selected based on their proteolytic capabilities and potential to generate peptides <10 kDa from sodium caseinate. All 15 isolates produced a zone size ≧4 mm in diameter size indicative of proteolytic activity, and HPLC analysis of small-scale casein fermentations of all 15 isolates showed the most complex peptide profiles. Of the fifteen isolates selected, 9 were identified as distinct species of lactobacilli following PFGE and 16S rDNA sequencing (Table 1). 5 isolates, all of human adult faecal origin, showed between 99-100% homology with Enterococcus faecalis (using the BLAST program) and therefore were considered unsuitable for further use in large-scale fermentations, as enterococci are not generally recognised as safe (GRAS) (21). Isolate 3L7 and 33L1 (human infant faecal isolates) showed 100% homology (BLAST program) with E. hirae and Staphylococcus epidermis, respectively, and these were also eliminated from further study due to non-GRAS status. 3 of the 9 lactobacilli isolates were identified as L. acidophilus DPC6026, L. johnsonii DPC6092 and L. salivarius DPC6027 by PFGE (FIG. 1) and 16S-rDNA sequencing.

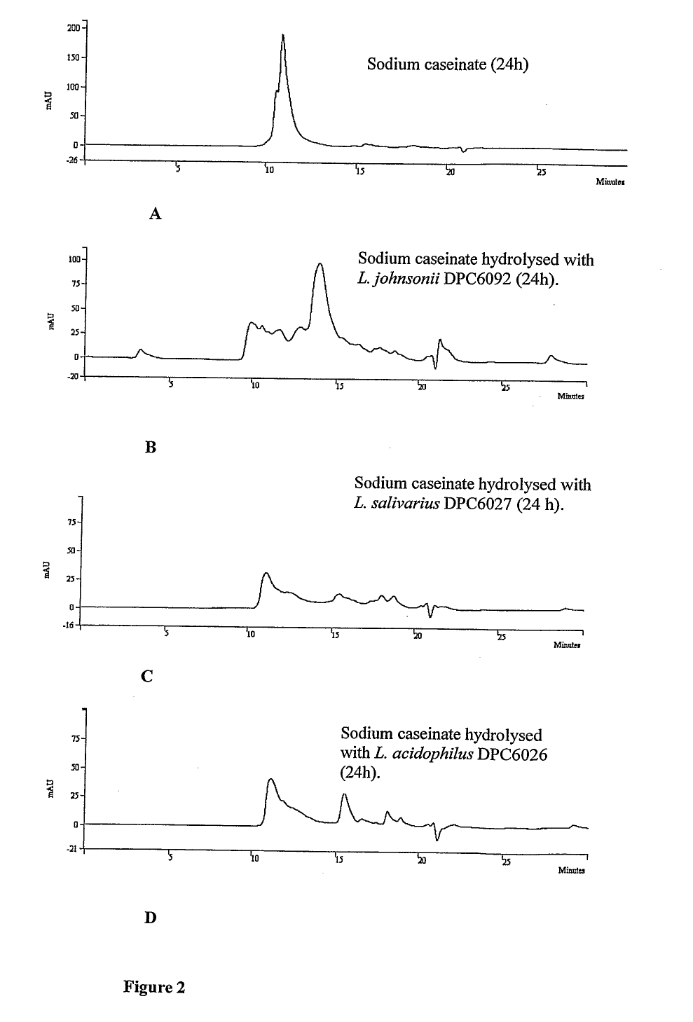

The peptide profiles obtained following incubation of L. acidophilus DPC6026, L. salivarius DPC6027 and L. johnsonii DPC6092 in sodium caseinate (2.5% w/v) after 24 h and subsequent filtration through a 10 kDa membrane is shown in FIG. 2. Fermentation of sodium caseinate with these 3 lactobacilli resulted in substantial degradation of the sodium caseinate (FIG. 2A) to lower molecular weight peptides (FIGS. 2B-2D) with L. acidophilus DPC6026 exhibiting the greatest degree of breakdown of sodium caseinate into smaller weight peptides (Table 1). L. acidophilus DPC6026 produced a fermentate where 60.74% of the peptides obtained were less than ≦0.5 kDa, 17.49% were peptides between 1-0.5 kDa and 11.57% peptides between 1-2 kDa. This strain produced more peptides between 0.5 kDa-2.0 kDa than L. johnsonii DPC6092, L. salivarius DPC6027 and L. animalis DPC6028 (Table 1). The HPLC peptide profiles produced by L. reuteri DPC6100, L. gasserri DPC6093, L. rhamnosus DPC6095 and L. brevi DPC6102 did not indicate breakdown of sodium caseinate to the same extent (HPLC profiles not shown) as the strain chosen (Table 1) and therefore were not chosen for further study. L. delbrueckii sp. bulgaricus DPC6104 was not chosen as its use for the generation of bioactive peptides has been documented previously (11).

### Detection of Antimicrobial Peptides

Sodium caseinate fermentates produced by Lactobacillus acidophilus DPC6026, L. salivarius DPC6027 and L. johnsonii DPC6092 were antibacterial fractions. However, only fractions generated from L. acidophilus DPC6026 were assayed for antibacterial activity, as this strain was characterised by the highest proteolytic activity of the three strains as shown from the chromatogram profiles shown (FIG. 2). Antimicrobial activity in the crude fermentates, using either the 96-well plate assay or the well diffusion assay against the strains E. coli DPC6053, Listeria innocua DPC3306, E. sakazakii 5920 (ATCC12868) and E. sakazakii 8272 (NCTC8155) was not detected. The sodium caseinate fermentate produced by L. acidophilus DPC6026 was filtered through a size-exclusion S1Y10 10 kDa spiral cartridge filter to obtain a permeate containing peptides <10 kDa. 72 fractions of the filtered sodium caseinate fermentate produced by L. acidophilus DPC6026 were collected by RP-HPLC and assayed for antibacterial activity against E. coli DPC6053, E. sakazakii 5920 (ATCC12868), E. sakazakii 8272 (NCTC8155) and L. innocua DPC3306 by a well diffusion assay. The antimicrobial activity of these fractions against E. coli DPC6053 and L. innocua DPC3306 was also assayed by measuring the OD₆₀₀ using the 96-well plate assay method. Three fractions, A1-45, A1-49, and A1-54 had the most potent antibacterial activity and had peptide concentrations of 0.554 mg/ml, 0.5 mg/ml and 1.24 mg/ml respectively. All these fractions exhibited inhibitory activity against the test strain E. coli DPC6053 at these concentrations, respectively.

### Purification, Sequencing and Characterisation of Peptides

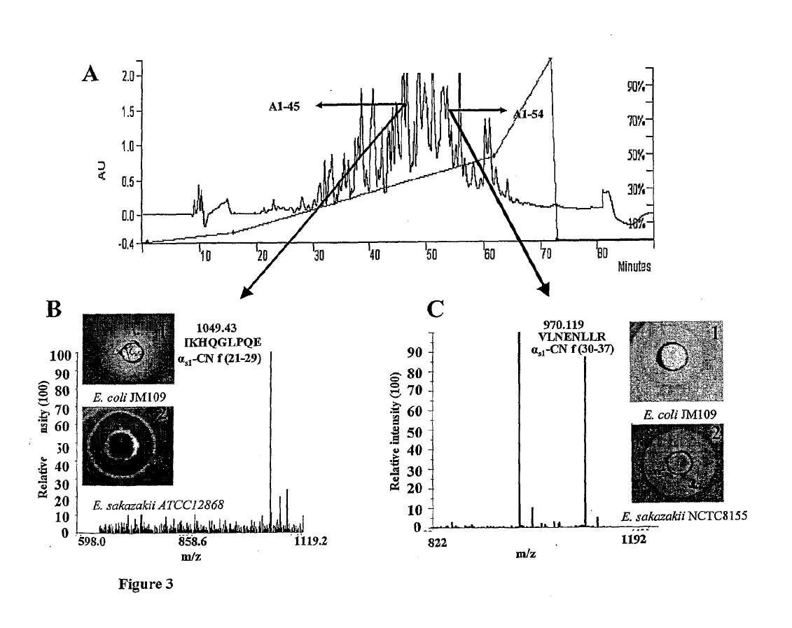

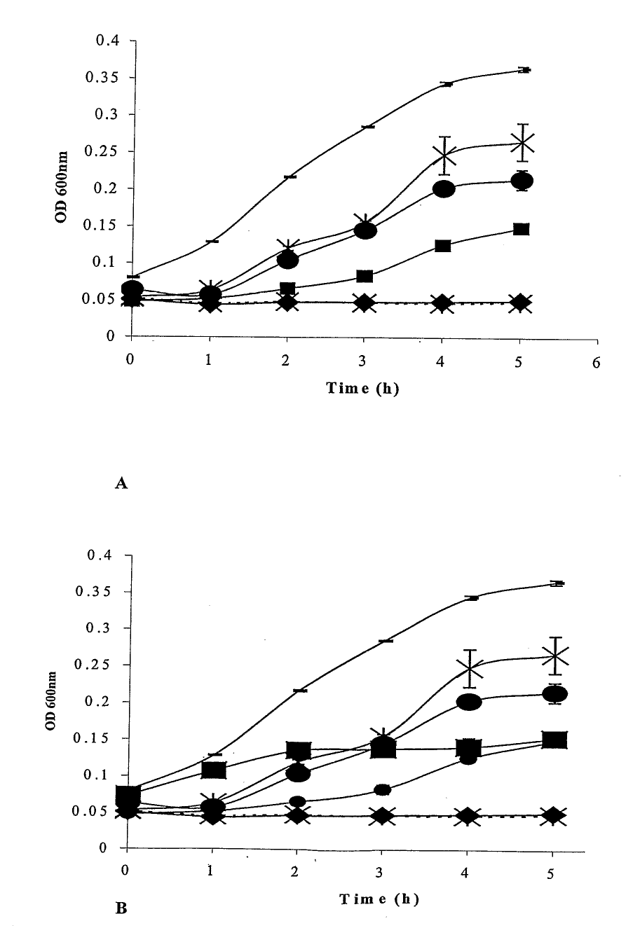

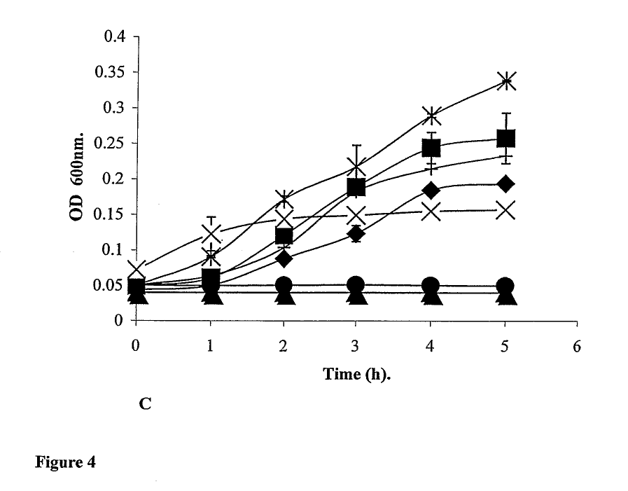

The peptide mixtures in fractions A1-45, A1-49 and A1-54 were subjected to mass spectrum analysis and Edman degradation. The amino-acid composition was also determined by sequencing (Aberdeen Proteome Facility). Fraction A1-45 was found to contain the peptide sequence IKHQGLPQE (SEQ ID NO: 1) (Table 2). This peptide sequence corresponds to αₛ₁-casein f(21-29). Fraction A1-49 contained the peptide sequence SDIPNPIGSENSEK (SEQ ID NO: 2) that corresponds to αₛ₁-casein f(183-207). A1-54 was found to contain the peptide sequence VLNENLLR (SEQ ID NO: 3) that corresponds to αₛ₁-casein f(30-37). The expected and calculated masses for each peptide are reported (Table 2). As determined by the agar well-diffusion assay, SEQ ID NO: 1 present in fraction 45 inhibited the indicator organism, E. coli DPC6053 at a concentration of 0.05 mM (FIG. 3 B). This peptide also showed inhibition against potentially pathogenic bacteria of clinical interest such as E. coli O157: H7 derivatives (E. coli DPC6054 and E. coli DPC6055), E. sakazakii 5920 (ATCC12868) (FIG. 3 B) at the same concentration (0.05 mM). SEQ ID NO: 3 present in fraction 54 inhibited the indicator organism, E. coli DPC6053 at a concentration of 0.22 mM (FIG. 3 C) and also against potentially pathogenic strains such as E. sakazakii DPC6091 at the same concentration (FIG. 3 C), while fraction A1-49 containing SEQ ID NO: 2 displayed only minor inhibitory activity against Listeria innocua DPC3306 but no activity against E. coli DPC6053 (Table 3).

### Chemical Synthesis of Peptides

Peptides identified from fractions that showed strong antibacterial activity and had a broad spectrum of activity were chemically synthesised to ensure that the sequence identified by MALDI-TOF analysis was the antibacterial sequence within the fractions. These were αₛ₁-CN f(21-29) (SEQ ID NO: 1), αₛ₁-CN f(30-37) (SEQ ID NO: 3) and αₛ₁-CN f(183-207) (SEQ ID NO: 2). Chemically synthesised peptides SEQ ID NO: 1 and SEQ ID NO: 3 inhibited the same microorganisms as the fractions containing the unpurified peptides. Synthesised peptide SEQ ID NO: 2 (present in fraction A1-49) displayed some inhibitory activity against Listeria innocua DPC3306 but no activity against E. coli DPC6053 (Table 3).

The minimum inhibitory concentration (MIC) of these peptides was determined using isracidin as a positive control. Isracidin inhibited E. coli DPC6053 at concentrations ranging from 0.05 mM to 1.9 mM (FIG. 4A). The MIC for isracidin was found to be 0.059 mM under the experimental conditions described while the MIC for SEQ ID NO: 1 was 0.078 mM shown to inhibit growth of E. coli DPC6053 (FIG. 4 B) and SEQ ID NO: 3 inhibited this microorganism at concentrations ranging from 0.22 mM to 1.2 mM. (FIG. 4 C). The MIC obtained for SEQ ID NO: 3 was 0.22 mM. Thus, the present invention has surprisingly produced three novel peptides all with antimicrobial properties. Isracidin was considered as a gold standard positive control: SEQ ID NO: 1 compares favourably with isracidin, whereas SEQ ID: NO 2 and SEQ ID NO: 3 are a factor less effective at the same concentration. However, notwithstanding the requirement for greater concentration, both SEQ ID NO: 2 and SEQ ID NO: 3 clearly demonstrated antimicrobial properties.

The concentrations require for SEQ ID NO: 2 and SEQ ID NO: 3 are within the normal concentration ranges for antimicrobial compounds. They compare well with, for example, Casocidin-1 and the peptides described in U.S. Pat. No. 6,579,849 “Antibiotic peptides from bovine milk”, which lists desired concentrations of 0.1 mg-1 mg of the composition. Floris et al., Current Pharmaceutical Design, 2003, 9, 1257-1275, state that isracidin “was found to inhibit the in vitro growth of lactobacilli and other Gram-positive bacteria, but only at relatively high concentrations (0.1-1 mg/mL).”

Under assay conditions, all the chemically synthesised peptides and crude fractions lost their antimicrobial activity when treated with Proteinase K. Using the computer program Expasy Peptide cutter (http://ca.expasy.org/cgi-bin/peptidecutter/peptidecutter.pl) it was found that peptide sequences IKHQGLPQE (SEQ ID NO: 1), VLNENLLR (SEQ ID NO: 3), SDIPIGSENSEK (SEQ ID NO: 2) are all hydrolysed by trypsin and chymotrypsin.

### Discussion

In this study, sodium caseinate was subjected to proteolysis using the proteolytic strains L. acidophilus DPC6026, L. salivarius DPC6027 and L. johnsonii DPC6092. The strain L. acidophilus DPC6026 was chosen for use in fermentations and peptides generated assayed for antimicrobial activity against potentially pathogenic strains such as E. coli JM109 DPC6053 and Enterobacter sakazakii DPC6090 (ATCC12868).

The peptides sequences IKHQGLPQE (SEQ ID NO: 1), VLNENLLR (SEQ ID NO: 3) and SDIPIGSENSEK (SEQ ID NO: 2) identified in this study have not been reported previously for antimicrobial activity (8, 47).

(Dziuba et al., 1999 (8)) makes reference to a bioactive database including a range of bioactive peptides derived from a variety of sources. Peptides included in the database are: antimicrobial peptides, angiotensin-I converting enzyme inhibitory peptides (ACE-I inhibitory peptides), opioid peptides and Bradykinin potentiating peptides. The peptide isracidin (Peptide Data ID 3035, name isracidin, sequence RPKHPIKHQGLPQEVLNENLLRP) used as the gold standard positive control against pathogenic bacteria in the experiments carried out is detailed in this database.

(Wang et al., 2004 (47)) details an Antimicrobial Peptide Database only, and only briefly mentions isracidin.

### Conclusion of Study

A known antimicrobial peptide from bovine milk protein is isracidin, αₛ₁-CN f1-23, with the primary amino acid sequence determined as R₁PKHPIKHQGLPQEVLNENLLRF₂₃ (26). This peptide has a broad spectrum of activity (23) and was used as a positive control in the current study because of the high degree of homology between it and SEQ ID NO: 1 and SEQ ID NO: 3. SEQ ID NO: 1 has nine residues in common with isracidin, while SEQ ID NO: 3 has eight residues in common with the C-terminal end of isracidin. Peptide SEQ ID NO: 1 exhibited a MIC of 0.078 mM, comparable with isracidin, which exhibited a MIC of 0.05 mM, and the more potent commercially available peptides Cecropin P1 and indolicidin (MIC of 0.05 mM). SEQ ID NO: 3 exhibited a MIC of 0.22 mM against E. coli DPC6053, comparable with the MIC of isracidin (0.05 mM). Peptides of the SEQ ID NO: 1 (IKHQGLPQE) contains a positive charge of +2, has a hydrophobic (isoleucine) end and a hydrophilic (glutamate) domain and displays better activity against Gram-negative bacteria such as E. sakazakii and E. coli than Gram-positive bacteria.

Antimicrobial peptides usually possess between 5 and 60 amino acids, have molecular masses of less than 10 kDa, are usually amphipathic, and usually have a broad spectrum of activity (Floris et al., Current Pharmaceutical Design 2003). SEQ ID NO: 3 (VLNENLLR) shares these traits.

It is known that the dual cationic and hydrophobic nature of peptides is important for the initial interaction between the peptide and the bacterial membrane. Positivity promotes interaction with the bacterial outer and cytoplasmic membranes (Wu et al., Journal of Biol. Chem., 1999). The strong positive charge of arginine (R) allow ID SEQ: 3 (VLNENLLR) to interact with the lipopolysaccharide region of Gram-negative bacteria and to penetrate the negatively charged cytoplasmic membrane as described by Hancock et al., 1999 Peptide Antibiotics, Antimicrobial agents and chemotherapy: 1317-1323.

SEQ ID NO: 3 also has a hydrophobic region (VL) and a hydrophilic region (R). It also displays more potent activity against the pathogenic Gram-negative bacteria than against Gram-positive strains such as L. innocua. Also, the chemically synthesised peptides exhibited more potent activity against the Gram-negative strains such as E. coli DPC6053 than against L. innocua DPC3306.

The proteinases of LAB have been shown to hydrolyse more than 40% of the peptide bonds of β-CN and αₛ₁-CN resulting in the generation of oligopeptides (22). The complex peptidases of LAB then act upon these oligopeptides (31). Minervini et al. reported the generation of ACE inhibitory peptides using a partially purified proteinase from L. helveticus PR4 (30). This group also identified an antimicrobial peptide from human β-CN f(184-210) that displayed activity against both Gram-positive and Gram-negative bacteria. Several casokinins derived from β-CN have been liberated by a cell-wall-associated serine-type proteinase of Lactobacillus helveticus CP790 (49). A cell-wall-bound proteinase from L. delbrueckii subsp. lactis ACA-DC178 liberated four peptides from β-CN (45), however; biological activity of these peptides were not reported.

L. acidophilus DPC6026 was chosen based on initial assays demonstrating its proteolytic ability against casein and therefore its potential to generate a large number of peptides with bioactivities such as antibacterial activity. Cecropin P1 was used as a control as it is a peptide isolated from the porcine small intestine and its spectrum of activity is chiefly against Gram-negative bacteria (5).

Chemically synthesised peptides of SEQ ID NO: 1, SEQ ID NO: 2 and SEQ ID NO: 3 and fractions containing these peptides were found to lose their antibacterial activity when treated with proteinase K. Trypsin and chymotrypsin inactivated all of the chemically synthesised peptides identified in the crude antibacterial fractions. Using the program Expasy Peptide cutter (http://ca.expasy.org/cgi-bin/peptidecutter/peptidecutter.pl) it was found that bovine αₛ₁-CN could be hydrolysed by a combination of three endopeptidases and proteinases to release the antibacterial peptides SEQ ID NO: 1 and SEQ ID NO: 3. A proline endopeptidase cleaves bovine αₛ₁-CN on the C-terminal side of Pro₂₀ and an endopeptidase restricts at the C-terminal side of Glu₂₉. This combination of enzymes would release the antibacterial peptide IKHQGLPQE (SEQ ID NO: 1) from bovine αₛ₁-CN. The presence of proline-specific peptidases is necessary for optimal growth of LAB because of their ability to degrade proline-rich oligopeptides from casein and proline-specific peptidases have previously been isolated from L. delbrueckii subsp bulgaricus (2). A combination of the Arg-C proteinase, which restricts bovine as1-CN at position Arg₃₇, and the endopeptidase that restricts bovine αₛ₁-CN at position Glu₂₉ would release the antimicrobial peptide VLNENLLR (SEQ ID NO: 3).

While not wishing to be bound by theory, it is believed that L. acidophilus DPC6026 used in fermentation under the conditions herein described (pH 7, maintained with 0.1M NaOH, 100 rpm, 37-42° C.) produces a novel collection of enzymes (proteinases and peptidases) such as Arg-C proteinase, capable of cleaving αₛ₁-CN into peptides SEQ ID NO 1 (IKHQGLPQE) and SEQ ID NO 3 (VLNENLLR) and SEQ ID NO 2 (SDIPIGSENSEK). This supposition is based on cleavage analysis of αₛ₁-Cn using the computer program http://ca.expasy.org/cgi-bin/peptidecutter/peptidecutter.pl and enzymes from the enzyme list provided that may cleave αₛ₁-CN to produce SEQ ID NO 1, SEQ ID NO 2 and SEQ ID NO 3.

Most antimicrobial peptides possess between 6 and 50 amino acid residues (48). The smallest antimicrobial peptide known at present is named hexapeptide with the sequence R₁RWQWR isolated from bovine lactoferricin (46). Most possess a positive charge of +2 or greater, and fold into amphipathic structures with both hydrophobic and hydrophilic domains (47). These features allow them to interact with the lipopolysaccharide region of Gram-negative bacteria and to penetrate the negatively charged cytoplasmic membranes (15).

From the sequence data alone, it can be difficult to predict either the activity of a peptide or the secondary structure that it may form (4). Peptides lacking disulfide bridges (as is the case with peptides IKHQGLPQE (SEQ ID NO: 1) and VLNENLLR (SEQ ID NO: 3) have random structures in water and only when they bind to a membrane or self-aggregate can they form a structure (10). However, some features of these peptides suggest that the mechanism of action may involve self-promoted uptake across the cytoplasmic membrane followed by interference with the cytoplasmic membrane barrier, a mechanism that has been described previously for cationic and neutral antimicrobial peptides (15, 48).

The features include (1) Positively charged peptides such as arginine contributing to the cationicity of the peptide which is important for the initial interaction with the bacterial cytoplasmic membrane. (2) Hydrophobicity—this is contributed to the peptide SEQ ID NO: 1 by the amino acid isoleucine and to peptide SEQ ID NO: 3 by the amino acids Valine (V) and Leucine (L). The present invention also provides for increasing the hydrophobicity of these peptides increase binding of the peptides to the membrane due to increased hydrophobic interactions between lipid acyl chains and the hydrophobic core.

### Conclusion

This study shows that the strain L. acidophilus DPC6026 is suitable for the generation of antimicrobial peptides from casein and that sodium caseinate fermentates produced with this strain of Lactobacillus may be considered as a component of functional foods with antibacterial benefits, as the MIC concentration of the antibacterial peptides are comparable with the commercially synthesised peptides (Cecropin P1) and the known antimicrobial peptide isracidin (22).

It also shows that bovine αₛ₁ casein may be considered as a precursor of the broad spectrum antibacterial peptides isolated here and finally that these peptides may have the potential to be used in infant formula products as an in-built protection system against pathogenic bacteria such as E. sakazakii.

Examples of a Method of Preparation of a Sodium Caseinate Powder with Anti-Enterobacter sakazakii Effects.

(1) Sodium caseinate substrate (5% w/v) maybe inoculated with (1% w/v) of L. acidophilus DPC 6026 and incubated at 37° C. for 24 hr with mixing at 100 rpm at constant pH 7 maintained via addition of 0.1 M NaOH. This fermentate can then be heated to 80° C. to inactivate the cultures which are then freeze dried to produce a fine powder.

(2) Sodium caseinate substrate (5% w/v) maybe inoculated with (1% w/v) of L. acidophilus DPC6026 and incubated at 37° C. for 24 hr with mixing at 100 rpm at constant pH 7 maintained via addition of 0.1 M NaOH.

(3) This fermentate may then be is then heated to 80° C. to inactivate the cultures and subsequently filtered through a size exclusion column 10 kDa to make a powder containing peptides less than 10 kDa. This downstream processing increases the activity of the active peptides. The 10 kDa membrane filtrate is freeze dried and stored at −20° C. and vacuum packed.

(4) Step (2) maybe followed as described above but a 3 kDa membrane filter maybe used to further increase the activity of the peptides SEQ ID NO; 1 and SEQ ID NO:3.

The above powders could be added at a concentration of 10%-15% to dried milk based infant formulas to prevent contamination with Enterobacter sakazakii and subsequently enteric colitis and meningitis caused by this pathogen.

The anti-Enterobacter sakazakii powder manufactured as above provides an internal and in-built protection mechanism against Enterobacter sakazakii and other pathogens, especially if added to casein based milk baby formula products.

In some embodiments, the peptides of the invention can be used separately or together as adjuvants for processes for the preparation of milk-based infant formula to safeguard against Enterobacter sakazakii contamination—a prominent cause of infection in immunocompromised and premature neonates.

The peptides of the invention can be used to contribute to bitter flavours in cheese manufacture as the first 9 residues of αₛ₁-CN (casein) are resistant to cleavage and have been documented as contributing to bitter flavours in cheese ripening (Pederson et al., 1999).

The production of αₛ₁-CN derived antimicrobial peptides by L. acidophilus DPC6026 has not been previously reported. The peptide sequences generated have not been found in various literature and database searches and are thought to be novel in this respect.

The words “comprises/comprising” and the words “having/including” when used herein with reference to the present invention are used to specify the presence of stated features, integers, steps or components but does not preclude the presence or addition of one or more other features, integers, steps, components or groups thereof.

### REFERENCES

1. Amsterdam, D. 1991. Antibiotics in Laboratory medicine, p. 72-78. In V. Lorian (ed.). Williams & Wilkins, Baltimore.
2. Atlan, D., P. Laloi, and R. Portalier. 1990. X-prolyldipeptidyl aminopeptidase of Lactobacillus delbrueckii subsp. bulgaricus: characterisation of the enzyme and isolation of deficient mutants. Applied and Environmental Microbiology. 56:2174-2179.
3. Bastian, E. D., and R. J. Brown. 1996. Plasmin in milk and dairy products: an update. International Dairy Journal 6:139-144.
4. Bello, J., H. R. Bello, and E. Granados. 1982. Conformation and aggregation of melittin: dependence on pH and concentration. Biochemistry 21:461-465.
5. Boman, H. G., B. Agerberth, and A. Boman. 1993. Mechanisms of action on Escherichia coli of cecropin P1 and PR-39, two antibacterial peptides from pig intestine. Infection and Immunity 61:2978-84.
6. Bujdakova, H., J. Hanzen, S. Jankovicova, J. Klimackova, M. Moravcikova, and M. Kettner. 2001. Occurrence and transferability of beta-lactam resistance in Enterobacteriaceae isolated in Children's University Hospital in Bratislava. Folia Microbiol. (Praha). 46:339-344.
7. de Palencia, F., P. C. Pelaez, C. Romero, and M. C. Martin-Hernandez. 1997. Purification and characterisation of the cell wall proteinase of Lactobacillus casei subsp. casei IFPL 731 isolated from raw goats milk cheese. Journal of Agricultural Food Chemistry 45:95-97.
8. Dziuba, J., P. Minkiewicz, D. Natecz, and A. Iwaniak. 1999. Database of biologically active peptide sequences. Nahrung 43:190-195.
9. El Soda, M., M. J. Desmazeaud, D. Le Bars, and C. Zevaco. 1986. Cell-wall associated proteinases in L. casei and L. plantarum. Journal of Food Protection. 49:361-365.
10. Falla, T. J., D. N. Karunaratne, and R. E. Hancocks. 1996. Mode of Action of the Antimicrobial peptide Indolicidin. The Journal of Biological Chemistry. 271:19298-19303.
11. Gobbetti, M., P. Ferranti, E. Smacchi, F. Goffredi, and F. Addeo. 2000. Production of angiotensin-I-converting-enzyme-inhibitory peptides in fermented milks started by Lactobacillus delbrueckii subsp. bulgaricus SS1 and Lactococcus lactis subsp. cremoris FT4. Appl Environ Microbiol 66:3898-904.
12. Gobbetti, M., F. Minervini, and C. G. Rizzello. 2004. Angiotensin I-converting-enzyme-inhibitory and antimicrobial bioactive peptides. International Journal of Dairy Technology. 57:173-188.
13. Gobbetti, M., L. Stepaniak, M. De Angelis, A. Corsetti, and R. Di Cagno. 2002. Latent bioactive peptides in milk proteins: proteolytic activation and significance in dairy processing. Crit. Rev Food Sci Nutr 42:223-39.
14. Hanberger, H., and L. E. Nilsson. 2001. High frequency of antibiotic resistance among gram-negative isolates in intensive care units at 10 Swedish Hospitals. Clinical Micro. Infect. 3:208-215.
15. Hancock, R. E. W., and D. S. Chapple. 1999. Peptide antibiotics. Antimicrobial agents and chemotherapy: 1317-1323.
16. Hickey, R. M., D. P. Twomey, R. P. Ross, and C. Hill. 2003. Production of enterolysin A by a raw milk enterococcal isolate exhibiting multiple virulence factors. Microbiology 149:655-664.
17. Hill, R. D., E. Lahav, and D. Givol. 1974. A rennin-sensitive bond in alpha-s1 B-casein. Journal of Dairy Research 41:147-53.
18. Hurley, M. J., L. B. Larsen, A. L. Kelly, and P. L. H. McSweeney. 2000. The milk acid proteinase cathepsin D: a review. International Dairy Journal. 10:673-681.
19. Kamysu, W., M. Okroj, and J. Lukasiak. 2003. Novel properties of antimicrobial peptides. Active Biochimica Polonica. 50.
20. Kandhai, M. C., M. W. Reji, G. Leon, M. Gorris, and M. van Schothorst. 2004. Occurrence of Enterobacter sakazakii in food production environments and households. The Lancet 363:5-9.
21. Korhonen, H., A. Pihlanto-Leppala, P. Rantamaki, and T. Tupasela. 2001. Milk protein derived bioactive peptides: Novel opportunities for health promotion. IDF Bulletin 363:17-26.
22. Kunji, E. R., G. Fang, C. M. Jeronimus-Stratingh, A. P. Bruins, B. Poolman, and W. N. Konings. 1998. Reconstruction of the proteolytic pathway for use of beta-casein by Lactococcus lactis. Molecular Microbiology. 27:1107-18.
23. Lahov, E., and W. Regelson. 1996. Antibacterial and immunostimulating casein-derived substances from Milk: casecidin, isracidin peptides. Fd. Chem. Toxic. 34:131-145.
24. Macart, M., and L. Gerbaut. 1982. An improvement of the Coomassie Blue dye binding method allowing an equal sensitivity to various proteins: application to cerebrospinal fluid. Clin Chim Acta 122:93-101.
25. Maeno, M., N. Yamamoto, and T. Takano. 1996. Identification of an Antihypertensive peptide from casein hydrolysate produced by a proteinase from Lactobacillus helveticus CP790. Journal of Dairy Science 79:1316-1321.
26. Malin, E., H. M. Alaimo, M. E. Brown, J. M. Aramini, and P. F. Fox. 2001. Solution structures of Casein Peptides: NMR, FTIR, CD, and molecular modeling studies of alpha s1 casein, 1-23. Journal of Protein Chemistry 20:391-404.
27. Martin-Hernandez, M., C. A. Alting, and F. Exterkate. 1994. Purification and characterisation of the mature, membrane-associated cell-envelope proteinase of Lactobacillus helveticus L89. Appl. Microbiol. Biotechnol. 40:828-834.
28. Maruyama, S., and H. Suzuki. 1982. A peptide inhibitor of angiotensin I-converting enzyme in the tryptic hydrolysate of casein. Agricultural and Biological Chemistry. 46:1393-1394.
29. Matar, C., J. G. LeBlanc, L. Martin, and G. Perdigon. 2003. Biologically Active Peptides Released in Fermented Milk Role and Functions., p. 177-199, Handbook of Fermented Functional Foods. CRC Press LLC.
30. Minervini, F., F. Algaron, C. G. Rizzello, P. F. Fox, V. Monnet, and M. Gobbetti. 2003. Angiotensin I-converting enzyme inhibitory and antibacterial peptides from Lactobacillus helveticus PR4 proteinase-hydrolysed caseins of milk from six species. Applied and Environmental Microbiology:5297-5305.
31. Mireau, I., E. R. Kunji, G. Venema, and J. Kok. 1997. Casein and peptide degradation in lactic acid bacteria. Biotech. Genet. Eng. Rev. 14:279-301.
32. Nakamura, Y., N. Yamamoto, K. Sakai, A. Okubo, S. Yamazaki, and T. Takano. 1995. Purification and characterization of angiotensin I-converting enzyme inhibitors from sour milk. J Dairy Sci 78:777-83.
33. Nazarowec-White, M., and J. M. Farber. 1997. Enterobacter sakazakii: a review. International Journal of Food Microbiology 34:103-113.
34. Nazarowec-White, M., and J. M. Farber. 1997. Thermal resistance of Enterobacter sakazakii in reconstituted dried-infant formula. Letters in Applied Microbiology 24:9-13.
35. Pag, U., M. Odenkoven, N. Papo, Z. Oren, Y. Shai, and H. G. Sahl. 2004. In vitro activity and mode of action of diastereomeric antimicrobial peptides against bacterial clinical isolates. Journal of Antimicrobial Chemotherapy. 53:230-239.
36. Palin, T., D. H. Kang, K. Schmidt, and D. Y. C. Fung. 2001. Detection of extracellular bound proteinase in EPS-producing lactic acid bacteria cultures on skim milk agar. Letters in Applied Microbiology 33:45-49.
37. Pihlanto-Leppala, A., J. Rokka, and H. Korhonen. 1998. Angiotensin I converting enzyme inhibitor peptides derived from Bovine Milk proteins. International Dairy Journal 8:325-331.
38. Pritchard, G. G., and T. Coolbear. 1993. The physiology and biochemistry of the proteolytic system in lactic acid bacteria. FEMS Microbiol. Rev. 12:179-206.
39. Quadri, L. E. N. 2002. Regulation of antimicrobial peptide production by autoinducer mediated quorum sensing in Lactic Acid Bacteria., p. 133-145. In G. Schaafsma (ed.), Lactic Acid Bacteria: Genetics, Metabolism and Application. Kluvwer Academic Publishers., Dordrecht.
40. Ryan, M. P., M. C. Rea, C. Hill, and R. P. Ross. 1996. An application in cheddar cheese manufacture for a strain of Lactococcus lactis producing a novel broad-spectrum bacteriocin, lacticin 3147. Applied and Environmental Microbiology. 62:612-619.
41. Sanders, W. E., and C. C. Sanders. 1997. Enterobacter spp.: Pathogens posied to flourish at the turn of the century. Clinical Microbiology Reviews, 10:220-241.
42. Simpson, P. J., C. Stanton, G. Fitzgerald, and R. P. Ross. 2003. Genomic diversity and relatedness of bifidobacteria isolated from a porcine cecum. J. Bacteriol. 185:2571-2581.
43. Sofia, V., F. Silva, and X. Malcata. 2005. Caseins as source of bioactive peptides. International Dairy Journal 15:1-15.
44. Stackebrandt, E., and B. M. Goebel. 1994. Taxonomic note: a place for DNA-DNA reassociation and 16S rRNA sequence analysis in the present species definition in bacteriology. Int. J. Syst. Bacteriol. 44:846-849.
45. Tsakalidou, E., R. Anastasiou, I. Vandenberghe, J. van Beeumen, and G. Kalantzopoulos. 1999. Cell-wall-bound proteinase of Lactobacillus delbrueckii subsp. lactis ACA-DC 178: characterization and specificity for beta-casein. Appl Environ Microbiol 65:2035-40.
46. Vogle, H. J., J. D. Schibli, W. Jing, F. R. Epand, and R. M. Epand. 2002. Towards a structure-function analysis of bovine lactoferricin and related tryptophan and arginine containing peptides. Biochem. Cell Biol. 80:49-63.
47. Wang, Z., and G. Wang. 2004. APD: the Antimicrobial Peptide Database. Nucleic Acids Research 32:590-592.
48. Wieprecht, T., M. Dathe, E. Krausse, M. Beyermann, W. L. Molloy, D. L. MacDonald, and M. Bienert. 1997. Modulation of membrane activity of amphipathic, antibacterial peptides by slight modifications of the hydrophobic moment. FEBS Letters 417:135-140.
49. Yamamoto, N., A. Akino, and T. Takano. 1994. Antihypertensive effect of the peptides derived from casein by an extracellular proteinase from Lactobacillus helveticus CP790. Biosci. Biotech. Biochem. 58:917-922.
50. Zevaco, C., and J. C. Gripon. 1988. Properties and specificity of a cell wall associated proteinase from Lactobacillus helveticus CP790. Le Lait 68:393-408.
51. Zucht, H. D., M. Raida, K. Adermann, H. J. Magert, and W. G. Forssman. 1995. Casocidin-I: a casein-alphaₛ₂ derived peptide exhibiting antibacterial activity. FEBS Letters 372:185-188.

TABLE 1
Sodium caseinate protein-derived hydrolysate fractions derived using
lactobacilli strains that generated peptide sizes of between <1-20 kDa.

| Lactobacilli | Peptide | Peptide | Peptide | Peptide | Peptide |
|---|---|---|---|---|---|
| strains used for in vitro hydrolyses of | Fraction | Fraction | Fraction | Fraction | Fraction |
| sodium caseinate (2.5% w/v). | 20-10 kDa % | 10-5 kDa % | 5-2 kDa % | 2-1 kDa % | <1 kDa % |
| L. acidophilus DPC6026 | 0.27 | 0.66 | 9.72 | 11.12 | 78.23 |
| L. johnsonii DPC6092 | 0.06 | 0.19 | 1.76 | 4.36 | 93.11 |
| L. salivarius DPC6027 | 0.08 | 0.32 | 2.19 | 3.56 | 93.85 |
| L. murinus DPC6028 | 0 | 0.04 | 2.36 | 4.71 | 92.89 |
| L. delbrueckii sp. bulgaricus DPC6105 | 0.02 | 0.18 | 3.98 | 14.49 | 81.34 |
| L. brevi DPC6102 | 0.01 | 2.62 | 1.59 | 2.66 | 93.12 |
| L. reuteri DPC6100 | 0.07 | 0.3 | 2 | 3.5 | 94.13 |
| L. gasserri DPC6093 | 0.01 | 0.14 | 2.58 | 5.09 | 92.19 |
| L. rhamnosus DPC6095 | 0.02 | 0.32 | 1.87 | 3.02 | 94.78 |

TABLE 2
16S rDNA sequencing of human infant and adult faecal
isolates and pig small intestinal isolates.

| | | | 16S rDNA |
|---|---|---|---|
| | | | sequencing % |
| Isolate | Isolate source | Strain | homology |
| 3L6 | Human adult faeces | L. gasserri DPC6093 | 100^{a} |
| 3L8 | Human adult faeces | E. faecalis DPC6094 | 100^{a} |
| 15L23 | Human adult faeces | L. rhamnosus DPC6095 | 99^{b} |
| 23L2 | Human infant faeces | E. faecalis DPC6096 | 100^{a} |
| 23L3 | Human infant faeces | E. faecalis DPC6097 | 100^{a} |
| 23L4 | Human infant faeces | E. faecalis DPC6098 | 99-100^{a} |
| 23L5 | Human infant faeces | E. faecalis DPC6099 | 100^{a} |
| 28L1 | Human infant faeces | L. reuteri DPC6100 | 98^{b} |
| 33L1 | Human infant faeces | S. epidermis DPC6101 | 100^{c} |
| 37L1 | Human infant faeces | L. brevi DPC6102 | 98^{b} |
| A1 | Pig small intestine | L. acidophilus DPC6026 | 99^{b} |
| B11 | Pig small intestine | L. johnsonii DPC6092 | 98^{b} |
| B24 | Pig small intestine | L. salivarius DPC6027 | 100^{b} |
| B28 | Pig small intestine | L. murinus DPC6028 | 98^{b} |
| 55 | Pig small intestine | L. delbrueckii sp. | 98^{b} |
| | | bulgaricus DPC6105 | |

DPC; Dairy Products Research Centre, Teagasc Moorepark, Fermoy, Co. Cork, Ireland.
^{a}Percentage homology with Enterococcus species.
^{b}Percentage homology with Lactobacillus species.
^{c}Percentage homology with Staphylococcus species.

TABLE 3
Sequences and corresponding casein (CN) fragments of peptides
contained in crude fractions from sodium caseinate
hydrolysates produced by L. acidophilus DPC6026.

| | | | A.A.^{a} | Calculated | Expected |
|---|---|---|---|---|---|
| Sequence and | | Overall | Chain | Mass | Mass^{b} |
| CN-Fragment | Fraction | Charge | length | (m/z) | (m/z) |
| IKHQGLPQE | A1-45 | Positive | 9 | 1049.177 | 1049.43 |
| αₛ₁-CN21-29 | | | | | |
| VLNENLLR | A1-54 | Positive | 8 | 970.119 | 970.119 |
| αs1-CNf 30-37 | | | | | |
| SDIPNPIGSENSEK | A1-49 | Positive | 14 | 1486.536 | 1486.7 |
| αs1-CNf 183-207 | | | | | |

^{a}One letter symbols are used for amino acids.
^{b}Averages masses are reported for the expected mass of each peptide.

TABLE 4
The inhibitory spectrum of pure peptides synthesized following fermentation of
L. acidophilus DPC6026 in sodium caseinate.

| | | Peptide | Peptide | Peptide |
|---|---|---|---|---|
| | Strain | IKHQGLPQE | VLNENLLR | SDIPIGSENSEK |
| Indicator species | details | αₛ₁-CN f(21-29) | αₛ₁-CN f(30-38) | αₛ₁-CN f(183-207) |
| Staphylococcus | DPC5246^{a} | − | − | − |
| aureus | | | | |
| Escherichia coli | DPC6053^{a} | +++ | +++ | − |
| JM109 | | | | |
| E. coli O157:H7 | DPC6054^{a} | +++ | +++ | − |
| E. coli O157:H7 | DPC6055^{a} | +++ | +++ | − |
| Enterobacter | DPC6090^{a} | +++ | +++ | − |
| sakazakii | | | | |
| ATCC12868 | | | | |
| E. sakazakii | DPC6091^{a} | +++ | +++ | − |
| NCTC8155 | | | | |
| Listeria innocua | DPC3306^{a} | ++ | ++ | + |
| Lactobacillus | DPC5383^{a} | +++ | +++ | − |
| bulgaricus | | | | |
| ATCC11842 | | | | |
| Streptococcus | DPC4069^{a} | ++ | ++ | − |
| mutans | | | | |

^{a}Dairy Products Research Centre, Cork, Ireland.
+++, Zone of inhibition >2.0 cm in diameter produced.
++, Zone of inhibition >1.5 cm in diameter produced.
+, Zone of inhibition >1.0 cm in diameter produced.
−, No zone detected.

1: An antimicrobial compound comprising one or more peptides selected from SEQ ID NO: 1, SEQ ID NO: 2, SEQ ID NO: 3, or any combinations thereof.

2: A biologically pure culture of Lactobacillus acidophilus, comprising:
strain DPC6026 deposited at the National Collection of Industrial and Marine Bacteria, Aberdeen, Scotland on 18^{th} Nov. 2005 under the accession number NCIMB 41354, capable of producing peptides having antimicrobial activity from milk or a milk product;
a derivative thereof;
a mutant thereof; or
any combination thereof.

3: A cell-free culture supernatant, or a fraction thereof, obtained from the culture according to claim 2.

4: A compound produced by DPC6026, wherein one or more antimicrobial peptides are produced by DPC6026 when cultured in milk or milk products.

5: The compound as claimed in according to claim 4, wherein the one or more antimicrobial peptides are selected from SEQ ID NO: 1, SEQ ID NO: 2, and SEQ ID NO: 3, or any combinations thereof.

6: An antimicrobial composition selected from an antimicrobial compound, a culture and a supernatant, produced by DPC6026 cultured in milk or a milk product under conditions that promote antimicrobial peptide production.

7: (canceled)

8: A method of preventing, treating, limiting microbial infection or limiting microbial contamination comprising the steps of administering a composition comprising a DPC6026 culture, a DPC6026 supernatant, an antimicrobial peptide made by DPC6026, or any combinations thereof to an animal or human.

9: The method according to claim 8 wherein the microbial infection or microbial contamination is a contamination or infection caused by Escherichia coli, E. sakazakii, Streptococcus mutans, Listeria innocua, Klebsiella spp, Staphylococcus carnosus or any combinations thereof.

10: The method according to claim 8 wherein the infection or contamination is mastitis or meningitis.

11: (canceled)

12: The method according to claim 8, wherein the composition is provided in a milk-based formula.

13: (canceled)

14: A method of inhibiting angiotensin-I converting enzyme, opioid modulation, immunomodulation or antithrombosis comprising the step of administering an effective amount of a composition comprising a DPC6026 culture, a DPC6026 supernatant, and an antimicrobial peptide made by DPC6026 cultured in milk or a milk product under conditions that promote antimicrobial peptide production to an animal or human.

15: A method of producing an anti-microbial peptide from milk or milk products comprising the steps of
adding a composition to milk or a milk product; wherein the composition comprises an antimicrobial compound, a culture, a supernatant or any combinations thereof produced by DPC6026 cultured in milk or a milk product under conditions that promote antimicrobial peptide production.

16: A foodstuff comprising an antimicrobial compound, a culture, a supernatant or any combinations thereof produced by DPC6026.

17: The foodstuff according to claim 16, wherein the foodstuff is selected from infant milk formula, milk powder, yoghurt, cheese, probiotic drink, baby food formula, diary-based drink, food supplement, or any combinations thereof.

18: A pharmaceutical composition comprising a therapeutically effective amount of a compound comprising an antimicrobial compound, a culture, a supernatant or any combinations thereof; wherein the compound is produced by DPC6026 cultured in milk or a milk product under conditions that promote the production of one or more antimicrobial peptide.

19: (canceled)


SEQUENCE LISTING (10 sequences)

SEQ ID NO: 1 - protein, 8 aa, Lactobacillus acidophilus
  misc_feature: Strain DPC6026
IKHQHPQE

SEQ ID NO: 2 - protein, 14 aa, Lactobacillus acidophilus
  MISC_FEATURE: Strain DPC6026
SDIPNPIGSENSEK

SEQ ID NO: 3 - protein, 8 aa, Lactobacillus acidophilus
  MISC_FEATURE: Strain DPC6026
VLNENLLR

SEQ ID NO: 4 - protein, 39 aa, Bos taurus
  MISC_FEATURE: Casocidin-1 f(164-179)
KTKLTEEEKNRLNFLKKISQRYQKFALPQYLKTVYQHQK

SEQ ID NO: 5 - protein, 16 aa, Bos taurus
  MISC_FEATURE: Casocidin-1 f(164-179)
KKTKLTEEEKNRLNFL

SEQ ID NO: 6 - protein, 21 aa, Bos taurus
  MISC_FEATURE: Casocidin-1 f(187-207)
QKFALPQYLKTVYQHQKAMKQ

SEQ ID NO: 7 - protein, 23 aa, Bos taurus
  MISC_FEATURE: Isracidin alpha-s1-casein peptide residue 1-23
RPKHPIKHQGLPQEVLNENLLRF

SEQ ID NO: 8 - protein, 9 aa, Bos taurus
  MISC_FEATURE: alpha-s1-casein f(1-9)
RPKHPIKHQ

SEQ ID NO: 9 - DNA, 18 bp, Artificial
  16S rDNA primer - CO1 for the 5′ end
agtttgatcctggctcag

SEQ ID NO: 10 - DNA, 15 bp, Artificial
  16S rDNA primer - CO2 for the 3′ end
taccttgttacgact